(19) United States
(12) Plant Patent Application Publication
(10) Pub. No.: US 2016/0073568 P1
(43) Pub. Date: Mar. 10, 2016

(54) Cannabis plant named 'MIDNIGHT'

(50) ;

(71) Applicant: Ytzchak COHEN, Tel Aviv (IL)

(72) Inventor: Ytzchak COHEN, Tel Aviv (IL)

(21) Appl. No.: 14/757,041

(22) Filed: Nov. 12, 2015

Related U.S. Application Data
(63) Continuation of application No. 14/193,228, filed on Feb. 28, 2014.
(60) Provisional application No. 61/768,565, filed on Feb. 25, 2013.

(51) Int. Cl. A01H 5/00

(52) U.S. Cl. PLT/258

(57) ABSTRACT

The disclosure relates to a new and distinct Cannabis Sativa L. plant named ‘Midnight’, having an almost equal ratio of Cannabidiol (CBD) concentration to Tetrahydrocannabinol (THC) concentration, (e.g., substantially close to 1), as illustrated and described herein.

### BACKGROUND

The disclosure is directed to a new and distinct cultivar of Cannabis Sativa plant named ‘Midnight, characterized by an almost equal or substantially the same amount of Cannabidiol (CBD) concentration to Tetrahydrocannabinol (THC) concentration.

### SUMMARY

Provided herein is a new and distinct Cannabis Sativa L. plant named ‘Midnight’, having an almost equal ratio of Cannabidiol (CBD) concentration to Tetrahydrocannabinol (THC) concentration, as illustrated and described herein.

### BRIEF DESCRIPTION OF THE DRAWINGS

FIG. 1 illustrates a single fully grown plant of the variety;

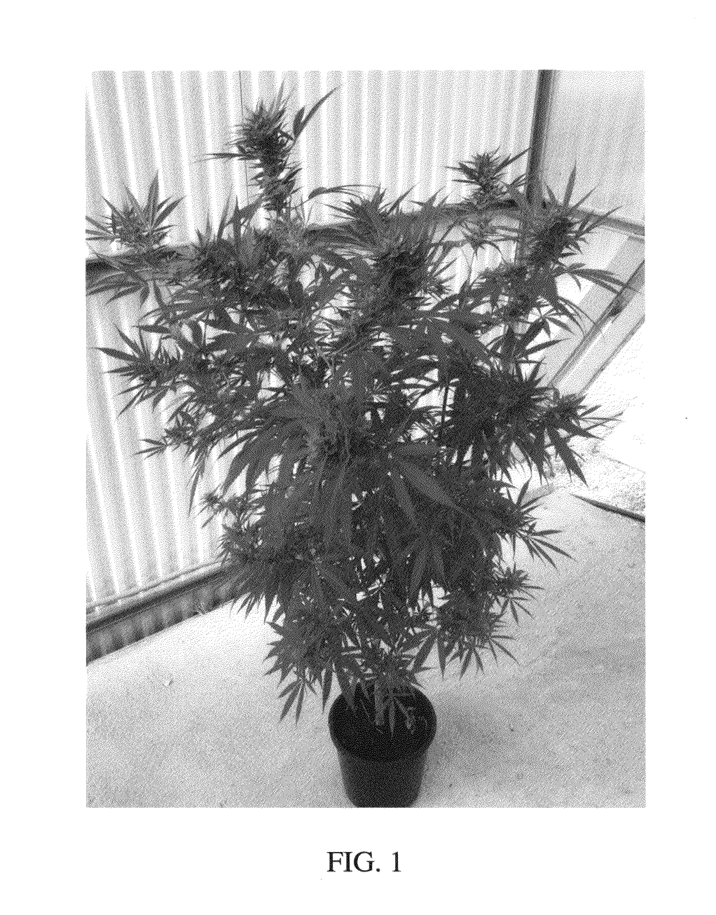

FIG. 2 illustrates a single leaf of the variety;

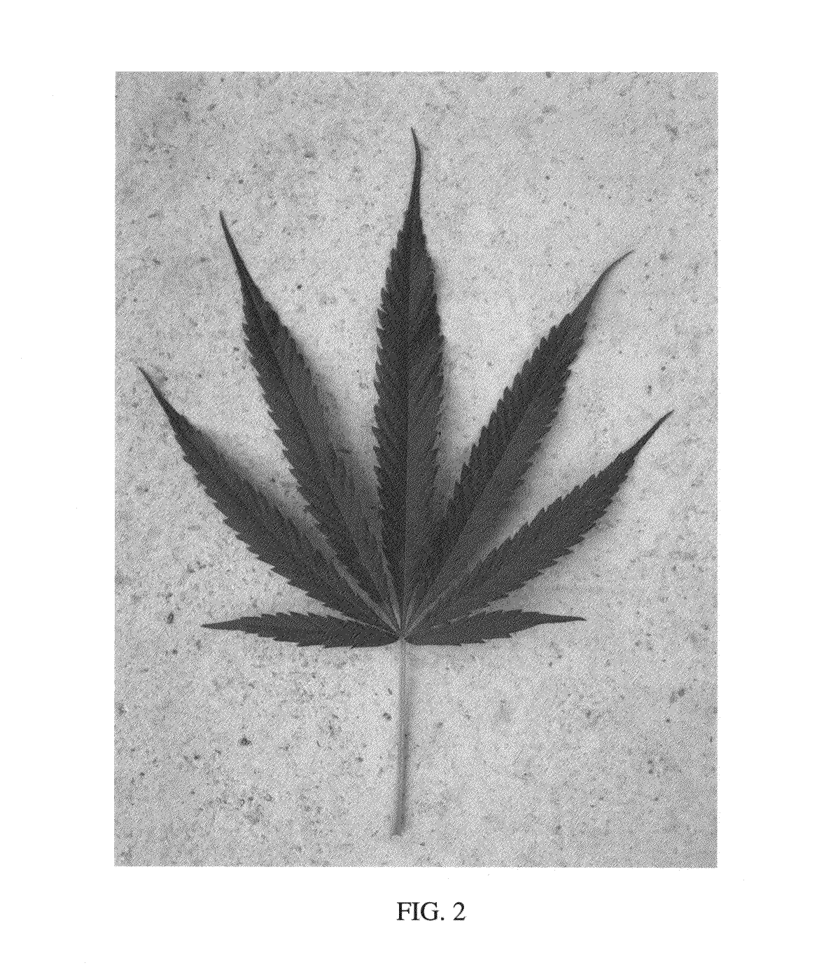

FIG. 3 illustrates a flowering plant in close-up; and

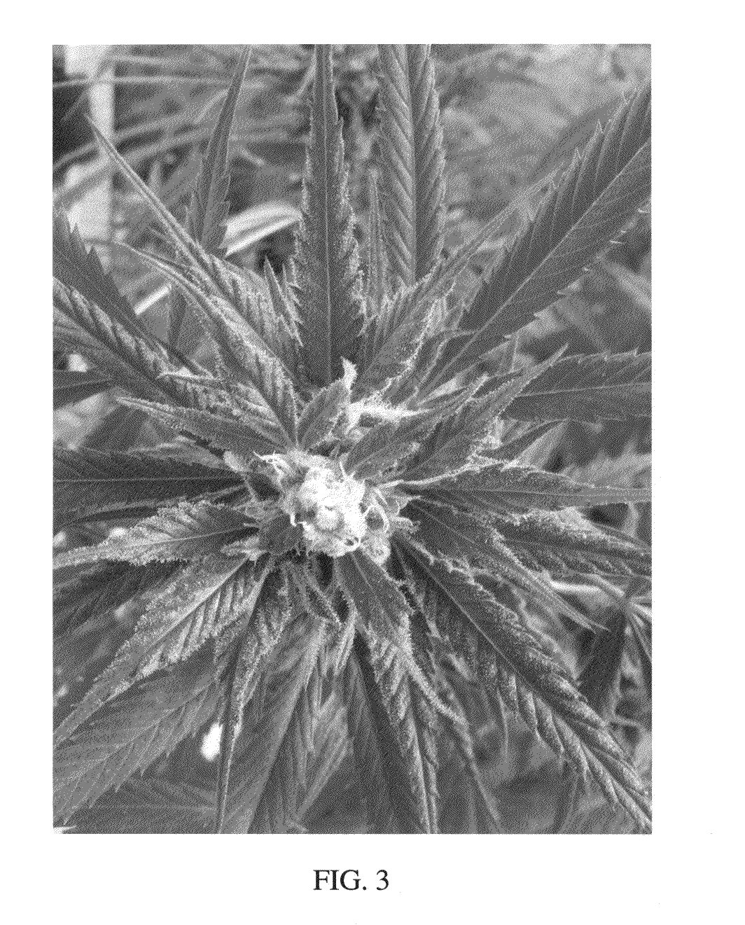

FIG. 4 illustrates a dried bud.

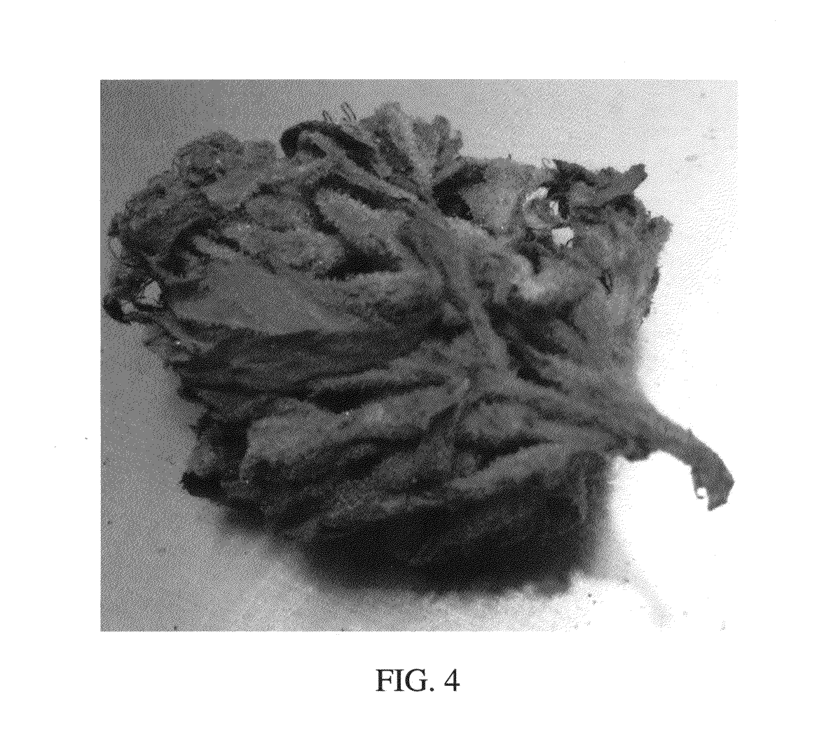

### DESCRIPTION

This invention relates to a new hybrid marijuana plant, Cannabis sativa L ssp. indica named Midnight. The Midnight is a medicinal variety of marijuana notable for an almost equal ratio of Cannabidiol (CBD) concentration to Tetrahydrocannabinol (THC) concentration. In other words, the ratio is substantially close to 1.

The present plant was developed over 3 years through selective breeding from parents of unknown provenance in Birya, Israel. Propagation occurs from seed, but large scale production generally occurs through asexual propagation using stem cuttings. The plant may be grown outside in a normal warm summer, including many locations below 50 N and virtually all climates below 45 N.

### PLANT

### Exposed Plant Structure

The Plant is an annual, dioecious plant with bush-like stature. The natural height at 5 months old of indoor growth is ˜150-160, and ˜170 cm outdoors

A detailed list of characteristics follows:
Botanical classification: Cannabis sativa L. ssp. indica
Parentage unknown:
Propagation: Stem cuttings.
Time to initiate roots — in summer:
Plant description: annual, dioecious flowering shrub; multi-stemmed; freely branching; removal of the terminal bud enhances lateral branch development.
Root description: Short taproot (less than 30 cm). Medium to fine, dense lateral roots; white in color, brownish when “potbound” (rootbound).
First year stems:
   Stem strength.—lateral stems are strong but benefit from being staked during flowering.
   First year stem color.—young stem: 144C; older stems: 144A (The Royal Horticultural Society Colour Chart, 1995 Ed.).
   First year stem diameter.—between about 2.5 cm and about 4.5 cm.
   First year trunk diameter.—between 7.0 cm and 8.0 cm at the soil line.
Foliage description:
   Branch description.—branches are short, densely branched with short, broad leaflets.
   Leaf arrangement.—palmately compound (digitate) leaves with 8 or 9 leaflets per leaf. This plant has an above-average number of leaves.
   Texture (upper and lower surfaces).—Upper surface scabrid with non-visible stiff hairs; lower surface more or less densely pubescent, covered with sessile glands.
   Color of emerging foliage (upper surface).—A cross between 144A and 144C (The Royal Horticultural Society Colour Chart, 1995 Edition.).
   Petiole length.—8.8 cm.
   Average internodes' length (lower half of plant).—5-6 cm, internodes' length (upper half of plant) 4 cm
   Venation pattern.—Palmately Compound (Digitate), with serrated leaflets. Each serration has a lateral vein extending to its tip from the central (primary) vein of the leaflet. In the middle leaflet, there is occasional double serration. From each lateral vein there is usually a single spur vein (sublateral vein) extending to the notch of each serration, but occasionally two spur veins.
Vegetative bud description: The lower flower buds can become too shaded by the leaves, resulting in a lower yield. This plant is particularly bushy for a sativa. The flower buds are highly resinous. Dried buds are dense and compact. There is a high density of trichomes; the visual appearance is ‘sparkling’, especially on the inner section of the dried buds. Dried buds are dense and compact. There is a high density of trichomes; the visual appearance is ‘sparkling’, especially on the inner section of the dried buds.
Flower description:
   Inflorescence.—Cone — or head-like, dense cluster of false spikes with solitary flowers instead of cymes. Enclosed by glandular, beaked bracteole. Flowers are often paired. Petals are absent. Calyxes are densely covered with trichomes, slight purpling of calyx and of primary veins of surrounding leafs.
   Wet flower color.—wet flower buds have many long white pistils, which become brown a week before harvest.
   Dried flower color.—Dried flower buds are a medium to dark green, with orange-brown hairs.
   Spike length.—ranges between 5 cm and 20 cm.
   Pistils description.—white, mid-sized, brown at maturation.
   Flowering time.—8 weeks.
Peduncles:
   Strength.—Strong, but can bend horizontally from weight of flower buds.
   Texture.—Moderately smooth, glabrous.
   Pedicels description.—Short, scabrid with sessile glands and visible hairs.

Seeds may be acquired through STS breeding. This involves inducing a female plant to become a male plant by altering its hormones using (in this case) silver nitrate and sodium thiosulfate. The male plant may then be used to pollinate a female of the same variety and collect the seeds. The relative proportion of male plants is low/medium.

The plant variety is similar to the Avidekel, both having high CBD content. The dried buds also have similar drying and storing properties including high mold resistance, high trichome density.

The inflorescences (flower buds) of this plant contain an almost equal ratio of CBD:THC (Approximately 12.4:12.7). When these two cannabinoids have an equal ratio, they work together synergistically for optimal medicinal benefits.

This strain is very versatile. It can be used to treat a wide range of health disorders. It has many beneficial medicinal qualities. Some uses include:
Stimulant
Anti-inflammatory
Pain management
Digestive disorders
Sleep disorders
Anti-nausea and vomiting (increases appetite)
Tourette syndrome
Parkinson's disease
Spasms
Post traumatic stress disorder (PTSD)

This plant requires frequent watering.

Rooting hormone and synthetic pesticides are used in propagation. This variety has high pest and disease tolerance

| USDA Plants Growth Habit Code | FB |
|---|---|
| Vigor | 5/5 |
| Productivity | Moderate to high productivity |
| Flowering timing | 9 Weeks |
| Flowering score | 7.5 |
| Branches | Weak |
| Fruit | Slightly swolen |
| THC level | 12.7% |
| CBD level | 12.4% |

A crop was grown having the following times of growth cycle operations:

| | |
|---|---|
| date of cutting/or from seeding | 21 Apr. 2011 |
| date transferred to cones pot of 0.8 liter | 27 Apr . 2011 |
| Date of moved to pot of 11 liters | 23 May 2011 |
| Date of taking clones | 03 Jun. 2011 |
| Date—start to flower | 20 Jun. 2011 |
| Ending flowering | 21 Aug. 2011 |
| wet flowers (grams) | 494.7 |
| wet leaves (grams) | 212.5 |
| wet branches (grams) | 211.8 |
| dry flowers (grams) | 137.8 |
| Mothers | 2 meter |
| Pot size | 25 L |

Permits are required for growth of medical marijuana in certain states in the US and Israel.

1. A new and distinct Cannabis Sativa L. plant named ‘Midnight’, having an almost equal ratio of Cannabidiol (CBD) concentration to Tetrahydrocannabinol (THC) concentration, as illustrated and described herein.
